(19) United States
(12) Patent Application Publication
(10) Pub. No.: US 2008/0014271 A1
(43) Pub. Date: Jan. 17, 2008

(54) NOVEL PHARMACEUTICAL COMPOSITIONS COMPRISING LEVETIRACETAM

(75) Inventors: Caroline Goffin, Bruxelles (BE); Michael Boonen, Verlaine, Chapon Seraing (BE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: UCB, S.A., Brussels (BE)

(21) Appl. No.: 11/681,473

(22) Filed: Mar. 2, 2007

Related U.S. Application Data
(60) Provisional application No. 60/807,526, filed on Jul. 17, 2006.

(30) Foreign Application Priority Data
Jul. 13, 2006 (EP) 06014537.2

(51) Int. Cl. A61K 31/40; A61K 9/42; A61P 25/08

(52) U.S. Cl. 424/476; 514/424

(57) ABSTRACT

The present invention relates to pharmaceutical compositions comprising levetiracetam as active ingredient, the invention relates specifically to a prolonged release formulation.

This application claims the benefit of U.S. provisional application 60/807,526, filed Jul. 17, 2006.

The present invention relates to a novel pharmaceutical composition comprising levetiracetam.

Levetiracetam or (S)-(−)-alpha-ethyl-2-oxo-1-pyrrolidine acetamide, a laevorotatory compound, is disclosed as a protective agent for the treatment and the prevention of hypoxic and ischemic type aggressions of the central nervous system in the European patent No. EP 0 162 036 B and has the following formula:

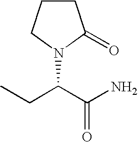

This compound is also effective in the treatment of epilepsy, a therapeutic indication for which it has been demonstrated that its dextrorotatory enantiomer (R)-(+)-alpha-ethyl-2-oxo-1-pyrrolidine acetamide completely lacks activity (A. J. Gower et al., Eur. J. Pharmacol., 222, 1992, 193-203).

A film-coated tablet containing 250 mg, 500 mg or 1000 mg levetiracetam is described in Rote Liste Service Gmbh “Rote Liste” 2003, 2002, ECV—Editio Cantor, Aulendorf, Germany. The ingredients are maize starch, povidone K30, talc, colloidal anhydrous silica, magnesium stearate, and in the coating hypromellose, macrogol 4000, titanium dioxide.

One of the objectives currently sought in the development of pharmaceutical compositions which can be administered orally is to control the release of pharmaceutically active substances so that they can be administered in a few daily doses, ideally in a single daily dose.

Indeed, the quantities of excipients necessary for adequate prolonged release of the active ingredient can prove to be too high and can make the production of the dosage form impossible or too costly. Moreover, in that case the tablet size may be too large so that the tablet cannot be swallowed.

In fact levetiracetam is a very soluble active ingredient, so it is difficult to slow down the release. Moreover another problem consists in the reduction of the release rate, while keeping a reasonable size for a high dose of very soluble active ingredient.

According to one aspect, the present invention relates to a pharmaceutical composition in the form of a tablet comprising, as active ingredient, levetiracetam and, as excipient within the core of the tablet, 5.0 to 59.0% per weight of at least one hydrophilic matrix agent, with respect to the total weight of the core of the tablet.

The term “active ingredient” as used herein is defined as a substance which has a therapeutic effect.

The amount of the active ingredient present in the pharmaceutical composition of the invention may vary depending on the mammal to which the compositions are administered and the disease to be treated.

The term “core of the tablet” as used herein is defined as the pharmaceutical composition without coating. All the percentages are given per weight of the total weight of the core of the tablet, except when it is written otherwise.

The term “hydrophilic matrix agent” as used herein is defined as a pharmaceutical acceptable excipient which generates a gel in contact with water. A “hydrophilic matrix agent” is a material that is a water dispersible rate controlling polymer. 3 types of water dispersible rate controlling polymer are available: hydrophilic, hydrophobic and inert polymers.

Examples of hydrophilic matrix agents which can be used according to the present invention are: cellulose derivatives (hydroxypropyl methylcellulose, hydroxyethyl cellulose, hydroxypropyl cellulose, methylcellulose and the like); noncellulose polysaccharides (galactomannans, guar gum, carob gum, gum arabic, sterculia gum, agar, alginates and the like); polyvinylpyrrolidone; polyvinylacetate; acrylic acid polymers, such as crosslinked acrylic acid-based polymers; and a mixture of two or more of the said agents. The hydrophilic matrix agents may be present in the form of a single compound or in the form of a mixture of compounds. The hydrophilic matrix agents preferably used according to the present invention are hydroxypropyl methylcelluloses, such as METHOCEL® K or E; polyvinylpyrrolidone; polyvinylacetate; a mixture of polyvinylpyrrolidone and polyvinylacetate, such as KOLLIDON SR®; and crosslinked acrylic acid-based polymers, such as CARBOPOL®. More preferably, the hydrophilic matrix agents are hydroxypropyl methylcelluloses, such as METHOCEL K or METHOCEL E.

In a preferred embodiment of the invention, the pharmaceutical composition comprises at least two hydrophilic matrix agents. In a more preferred embodiment of the invention, the pharmaceutical composition comprises hydroxypropyl methylcelluloses and a mixture of polyvinylpyrrolidone and polyvinylacetate, or hydroxypropylmethylcellulose and crosslinked polyacrylic acid polymers.

Usually, the pharmaceutical composition according to the present invention comprises 5.0 to 59.0% per weight of hydrophilic matrix agent with respect to the total weight of the core of the tablet.

Particularly, the pharmaceutical composition according to the present invention comprises 8.0 to 50.0% per weight of hydrophilic matrix agent.

Preferably, the pharmaceutical composition according to the present invention comprises 15.0 to 40.0% per weight of hydrophilic matrix agent, more preferably 20.0 to 30.0% per weight of hydrophilic matrix agent, most preferably 25.0 to 28.0% per weight of hydrophilic matrix agent with respect to the total weight of the core of the tablet.

Moreover, further to the hydrophilic matrix agent(s) inert and lipophilic matrix agents may be added to form a mixed matrix composed of a combination of water non-dispersible and water dispersible polymers. These inert and lipohilic excipients may be present in less than 35% per weight of the core of the tablet.

Consequently, the pharmaceutical composition of the invention may also comprise, as excipient within the core of the tablet, an inert matrix agent. Examples of inert matrix agent which can be used according to the present invention are excipients essentially belonging to the class of thermoplastic polymers. They are inert towards biological tissues, other excipients in the formulation and the active substance. They are insoluble and indigestible in the fluids of the gastrointestinal tract. Among these, there may be mentioned polyvinyl chloride, polyethylene, vinyl acetate/vinyl chloride copolymers, polymethylmethacrylates, polyamides, silicones, ethyl cellulose, polystyrene and the like. Preferably the inert matrix agent used according to the invention is polyvinyl chloride such as the compound sold under the trademark PEVIKON®.

The pharmaceutical composition according to the present invention may comprise 0.0 to 35.0% per weight of inert matrix agent with respect to the total weight of the core of the tablet. Preferably, the pharmaceutical composition according to the invention does not comprise any inert matrix agent.

Moreover, further to the hydrophilic matrix agent(s) the core of the tablet may contain lipophilic matrix agents to form a mixed matrix composed of a combination of water non-dispersible and water dispersible polymers.

The pharmaceutical composition of the invention may also comprise, as excipient, a lipophilic matrix agent. Examples of lipophilic matrix agent which can be used according to the present invention are excipients of four types of fatty excipients: glycerides (mono-, di- or triglycerides: stearin, palmitin, laurin, myristin, hydrogenated castor or cottonseed oils, glyceryl palmitostearate (Precirol) and the like), fatty acids and alcohols (stearic, palmitic or lauric acids; stearyl, cetyl or cetostearyl alcohols, and the like), fatty acid esters (monostearates of propylene glycol and of sucrose, sucrose distearate and the like) and waxes (white wax, cachalot wax and the like) or mixtures of two or more of them. Preferably the lipophilic matrix agent used according to the invention is glyceryl palmitostearate, such as the compound sold under the trademark Precirol®.

The pharmaceutical composition according to the present invention may comprise 0.0 to 35.0% per weight of lipophilic matrix agent with respect to the total weight of the core of the tablet. Preferably, the pharmaceutical composition according to the invention does not comprise any lipophilic matrix agent.

The pharmaceutical composition of the invention may also comprise a gliding agent, as excipient within the core of the tablet.

The term “gliding agent” as used herein is defined as an agent improving the fluidity of the powder and thus the filling of the granulation machine of the tablet press. The gliding agent may be present in the pharmaceutical composition in the form of a single compound or in the form of a mixture of compounds.

Examples of gliding agents are talc, starches, stearic acid and anhydrous colloidal silica. Preferred gliding agent according to the present invention is anhydrous colloidal silica, such as AEROSIL 200®.

Usually, the pharmaceutical composition according to the present invention comprises 0.0 to 3.0% per weight of gliding agent Preferably, the pharmaceutical composition according to the present invention comprises 0.3 to 2.5% per weight of gliding agent, more preferably 0.4 to 2.0% per weight of gliding agent, most preferably 0.5% per weight of gliding agent with respect to the total weight of the core of the tablet.

The pharmaceutical composition of the invention may also comprise a lubricant, as excipient within the core of the tablet.

The term “lubricant” as used herein is defined as an agent able to decrease adhesion of a powder to punches and friction between particles. The lubricant may be present in the pharmaceutical composition in the form of a single compound or in the form of a mixture of compounds.

Examples of lubricants are talc, magnesium stearate, calcium stearate or macrogol (also referred to as polyethylene glycol or PEG).

### Preferred lubricant according to the present invention is magnesium stearate and macrogol 6000.

As will be understood by the person skilled in the art, the number “6000” after polyethylene glycol refers to the average molecular weight of the polyethylene glycol.

In a preferred embodiment of the invention, the pharmaceutical composition comprises at least two lubricants. In a more preferred embodiment of the invention, the pharmaceutical composition comprises magnesium stearate and macrogols 6000.

Usually, the pharmaceutical composition according to the present invention comprises 0.0 to 5.50% per weight of lubricant with respect to the total weight of the core of the tablet.

Particularly, the pharmaceutical composition according to the present invention comprises 0.0 to 3.50% per weight of lubricant with respect to the total weight of the core of the tablet.

Preferably, the pharmaceutical composition according to the present invention comprises 0.4 to 1.30% per weight of lubricant with respect to the total weight of the core of the tablet.

Usually, the present invention relates to a pharmaceutical composition comprising levetiracetam as active ingredient and

5.0 to 59.0% per weight of hydrophilic matrix agent,

0.3 to 3.0% per weight of gliding agent, and

0.0 to 5.50% per weight of lubricant,

with respect to the total weight of the core of the tablet.

Particularly, the present invention relates to a pharmaceutical composition comprising levetiracetam as active ingredient and

8.0 to 50.0% per weight of hydrophilic matrix agent,

0.3 to 2.5% per weight of gliding agent, and

0.0 to 3.5% per weight of lubricant,

with respect to the total weight of the core of the tablet.

Preferably, the present invention relates to a pharmaceutical composition comprising levetiracetam as active ingredient and

15.0 to 40.0% per weight of hydrophilic matrix agent,

0.4 to 2.0% per weight of gliding agent, and

0.4 to 1.30% per weight of lubricant,

with respect to the total weight of the core of the tablet.

More preferably, the present invention relates to a pharmaceutical composition comprising levetiracetam as active ingredient and

20.0 to 30.0% per weight of hydrophilic matrix agent,

0.5% per weight of gliding agent and

0.4 to 1.30% per weight of lubricant

with respect to the total weight of the core of the tablet.

In a particular embodiment, the present invention relates to a pharmaceutical composition comprising levetiracetam as active ingredient and

5.0 to 59.0% per weight of hydroxypropylmethylcellulose,

0.0 to 3.0% per weight of anhydrous colloidal silica,

0.0 to 5.0% per weight of polyethylene glycol 6000, and

0.0 to 1.0% per weight of magnesium stearate,

with respect to the total weight of the core of the tablet.

Usually, in a particular embodiment, the present invention relates to a pharmaceutical composition comprising levetiracetam as active ingredient and

5.0 to 59.0% per weight of hydroxypropylmethylcellulose,

0.3 to 2.5% per weight of anhydrous colloidal silica,

0.5 to 5.0% per weight of polyethylene glycol 6000, and

0.0 to 1.0% per weight of magnesium stearate,

with respect to the total weight of the core of the tablet.

Particularly, in a particular embodiment, the present invention relates to a pharmaceutical composition comprising levetiracetam as active ingredient and

8.0 to 50.0% per weight of hydroxypropylmethylcellulose,

0.3 to 2.5% per weight of anhydrous colloidal silica,

0.5 to 1.5% per weight of polyethylene glycol 6000, and

0.0 to 0.5% per weight of magnesium stearate,

with respect to the total weight of the core of the tablet.

Preferably, in a particular embodiment, the present invention relates to a pharmaceutical composition comprising levetiracetam as active ingredient and

15.0 to 40.0% per weight of hydroxypropylmethylcellulose,

0.4 to 2.0% per weight of anhydrous colloidal silica,

0.7 to 1.5% per weight of polyethylene glycol 6000, and

0.1 to 0.3% per weight of magnesium stearate,

with respect to the total weight of the core of the tablet.

More preferably, in a particular embodiment, the present invention relates to a pharmaceutical composition comprising levetiracetam as active ingredient and

20.0 to 30.0% per weight of hydroxypropylmethylcellulose,

0 to 25% per weight of inert or lipohilic matrix agent,

0.5% per weight of anhydrous colloidal silica,

1.0% per weight of polyethylene glycol 6000, and

0.25% per weight of magnesium stearate,

with respect to the total weight of the core of the tablet.

More preferably, in a particular embodiment, the present invention relates to a pharmaceutical composition comprising levetiracetam as active ingredient and

20.0 to 30.0% per weight of hydroxypropylmethylcellulose,

0.5% per weight of anhydrous colloidal silica,

1.0% per weight of polyethylene glycol 6000, and

0.25% per weight of magnesium stearate,

with respect to the total weight of the core of the tablet.

The best results have been obtained with to a pharmaceutical composition consisting of levetiracetam as active ingredient and

20.0 to 30.0% per weight of hydroxypropylmethylcellulose,

0.5% per weight of anhydrous colloidal silica,

1.0% per weight of polyethylene glycol 6000, and

0.25% per weight of magnesium stearate,

with respect to the total weight of the core of the tablet.

In a further particular embodiment, the present invention relates to a pharmaceutical composition comprising 30.0 to 85.0% per weight of levetiracetam, with respect to the total weight of the core of the tablet.

Usually, in this further particular embodiment, the present invention relates to a pharmaceutical composition comprising 35.0 to 83.0% per weight of levetiracetam with respect to the total weight of the core of the tablet.

Particularly, in this further particular embodiment, the present invention relates to a pharmaceutical composition comprising 36.0 to 80.0% per weight of levetiracetam with respect to the total weight of the core of the tablet.

Preferably, in this further particular embodiment, the present invention relates to a pharmaceutical composition comprising 38.0 to 78.0% per weight of levetiracetam with respect to the total weight of the core of the tablet.

More preferably, in this further particular embodiment, the present invention relates to a pharmaceutical composition comprising 45.0 to 75.0% per weight of levetiracetam, with respect to the total weight of the core of the tablet.

In one embodiment of the present invention, the sum of hydrophilic matrix agent, gliding agent, and lubricant present in the pharmaceutical composition comprising levetiracetam as active ingredient is less than or equal to 70.0% per weight, preferably less than or equal to 35.0% per weight, more preferably less than or equal to 30.0% per weight with respect to the total weight of the core of the tablet.

Most preferably, the sum of hydroxypropylmethylcellulose, anhydrous colloidal silica, polyethylene glycol 6000, and magnesium stearate present in the pharmaceutical composition comprising levetiracetam according to the present invention is less than 28.6% per weight with respect to the total weight of the core of the tablet.

Last values for the sum of hydrophilic matrix agent, gliding agent and lubricant present the further advantage of reducing the size and weight of the pharmaceutical composition for a given quantity of active ingredient thereby increasing the ease of administration to a patient.

The pharmaceutical composition according to the present invention is preferably administered orally.

The pharmaceutical composition according to the present invention is preferably in the form of a solid, more preferably in the form of a tablet.

The tablet may be uncoated or coated with a coating agent.

In one embodiment, the pharmaceutical composition according to the present invention comprises 1.0 to 6.0% per weight of coating agent, preferably 2.0 to 5.0% per weight of coating agent, more preferably 2.5 to 4.5% per weight of coating agent, most preferably 2.9% per weight of coating agent with respect to the total weight of the pharmaceutical composition.

Examples of coating agents are hydroxypropylmethylcellulose, polyvinyl alcohol and methacrylic acid-alkyl acrylate copolymers.

Preferred coating agents are polyvinyl alcohol aqueous dispersions.

More preferred coating agent according to the present invention is Opadry®. An example of Opadry® is Opadry® 85F18422.

The coating agent preferably comprises polyvinyl alcohol (PVA) which coating agent ensures a better gliding of the tablets upon packaging. More preferably, the coating agent comprises partially hydrolyzed polyvinyl alcohol.

The presence of polyvinyl alcohol in the coating agent may also ensure a better adhesion of the coating to the tablet. Moreover, higher concentrations of coating agents in the aqueous suspension may be used.

In another embodiment, the pharmaceutical composition according to the present invention comprises 1.0 to 6.0% per weight of coating agent comprising polyvinyl alcohol, preferably 2.0 to 5.0% per weight of coating agent comprising polyvinyl alcohol, more preferably 2.5 to 4.5% per weight of coating agent comprising polyvinyl alcohol, most preferably 2.9% per weight of coating agent comprising polyvinyl alcohol with respect to the total weight of the pharmaceutical composition.

In this embodiment, the polyvinyl alcohol is preferably partially hydrolyzed.

In a particular embodiment according to the present invention, the sum of hydrophilic matrix agent, gliding agent, lubricant and coating agent present in the pharamaceutical composition comprising levetiracetam as active ingredient is less than or equal to 70% per weight, preferably less than or equal to 35% per weight, more preferably less than or equal to 30% per weight with respect to the total weight of the pharmaceutical composition.

In the above mentioned pharmaceutical compositions, Opadry® preferably comprises polyvinyl alcohol. More preferably, Opadry® comprises partially hydrolyzed polyvinyl alcohol.

In another particular embodiment, the sum of anhydrous colloidal silica, polyethylene glycol 6000, magnesium stearate and Opadry® in the pharmaceutical composition comprising levetiracetam is less than 32% per weight with respect to the total weight of the pharmaceutical composition.

Optionally, the pharmaceutical composition according to the present invention may contain a diluent or filler, such as starch.

Optionally, the pharmaceutical composition according to the present invention may contain a sweetening agent such as sucrose or saccharine, a coloring agent or a flavoring agent.

Optionally, the pharmaceutical composition according to the present invention may comprise a taste-masking agent.

Preferably, the pharmaceutical composition according to the present invention comprises a coating agent which has taste-masking properties.

The pharmaceutical composition of the invention can be manufactured by any process according to conventional methods known to the man skilled in the art. Examples of processes are direct compression, dry granulation, wet granulation, melt granulation.

Preferably, the process comprises a further coating step in which water, preferably purified water, is added to the coating agent and resulting suspension is sprayed on the core of the tablet.

Preferred coating agent is Opadry®. More preferred coating agent is Opadry® 85F18422. Most preferred coating agent is polyvinyl alcohol.

Specific formulations are as follows:
A tablet comprising 500 mg of Levetiracetam, 193.00 mg of hydroxypropyl methylcellulose (e.g. Methocel K15M CRP), 3.5 mg of an anhydrous colloidal silica (e.g. Aerosil 200) and 3.50 mg of magnesium stearate.
A tablet comprising 500 mg of Levetiracetam, 96.50 mg of hydroxypropyl methylcellulose (e.g. Methocel K15M CRP), 96.50 mg of a mixture comprising polyvinylacetate and polyvinylpyrrolidinone (e.g. Kollidon SR), 3.5 mg of an anhydrous colloidal silica (e.g. Aerosil 200) and 3.50 mg of magnesium stearate.
A tablet comprising 500 mg of Levetiracetam, 158.00 mg of hydroxypropyl methylcellulose (e.g. Methocel K15M CRP), 35 mg of a cross-linked acrylic acid based polymer (e.g. Carbopol 71G), 3.5 mg of an anhydrous colloidal silica (e.g. Aerosil 200) and 3.50 mg of magnesium stearate.
A tablet comprising 500 mg of Levetiracetam, 187.75 mg of hydroxypropyl methylcellulose (e.g. Methocel K15M Premium CR/EP), 7 mg of a PEG, 3.5 mg of an anhydrous colloidal silica (e.g. Aerosil 200) and 1.75 mg of magnesium stearate.

### Preferably the above described 4 tablets are coated with a polyvinyl alcohol, e.g. with Opadry®.

In another aspect the present invention relates to a pharmaceutical composition comprising levetiracetam useful for the treatment or prevention of a disease.

By the term “disease”, we understand a disease selected from the group consisting of epileptogenesis, seizure disorders, convulsions, Parkinson's disease, dyskinesia induced by dopamine replacement therapy, tardive dyskinesia induced by administration of neuroleptic drugs, Huntington Chorea, and other neurological disorders including bipolar disorders, mania, depression, anxiety, attention deficit hyperactivity disorder (ADHD), migraine, trigeminal and other neuralgia, chronic pain, neuropathic pain, cerebral ischemia, cardiac arrhythmia, myotonia, cocaine abuse, stroke, myoclonus, tremor, essential tremor, simple or complex tics, Tourette syndrome, restless leg syndrome and other movement disorders, neonatal cerebral haemorrhage, amyotrophic lateral sclerosis, spasticity and degenerative diseases, bronchial asthma, asthmatic status and allergic bronchitis, asthmatic syndrome, bronchial hyperreactivity and bronchospastic syndromes as well as allergic and vasomotor rhinitis and rhinoconjunctivitis.

The term “treatment” as used herein, includes curative treatment and prophylactic treatment.

By “curative” is meant efficacy in treating a current symptomatic episode of a disorder or condition.

By “prophylactic” is meant prevention of the occurrence or recurrence of a disorder or condition.

The present invention concerns also a method for treatment of a human patient by using the pharmaceutical composition.

The present invention concerns also the pharmaceutical composition for use as a medicament for curing the said disease.

The present invention concerns also the use of the pharmaceutical composition for the manufacture of a medicament for a therapeutic application in the said disease.

Preferably said disease is selected from the group consisting essentially of epilepsy, Parkinson's disease, dyskinesia, migraine, tremor, essential tremor, bipolar disorders, chronic pain, neuropathic pain, or bronchial, asthmatic or allergic conditions. More preferably said disease is epilepsy.

The present invention concerns also a method for manufacturing a medicament intended for therapeutic application in the said disease, characterized in that the pharmaceutical composition according to the present invention is used.

The present invention is also directed to methods of treating humans to alleviate disease by the administration of the pharmaceutical composition.

The following examples are provided for illustrative purposes only and are not intended, nor should they be construed, as limiting the invention in any manner. Those skilled in the art will appreciate that routine variations and modifications of the following examples can be made without exceeding the spirit or scope of the invention.

### EXAMPLES

### Example 1

### Formulations with a 24 h In Vivo Release of Levetiracetam

Tablets A, B, and C are prepared by direct compression process according to the invention with the following core compositions (Table 1). The cores are coated by an aqueous dispersion of polyvinyl alcohol cellulose.

TABLE 1
Core compositions of tablets A, B and C.

| | Tablet A | | Tablet B | | Tablet C | |
|---|---|---|---|---|---|---|
| Components | mg | % | mg | % | mg | % |
| Levetiracetam | 500.00 | 71.4 | 500.00 | 71.4 | 500.00 | 71.4 |
| Methocel K 15 MCR | 193.00 | 27.6 | 96.50 | 13.8 | 158.00 | 22.6 |
| Kollidon SR | — | — | 96.50 | 13.8 | — | — |
| Carbopol 71 G | — | — | — | — | 35.00 | 5.0 |
| Aerosil 200 | 3.50 | 0.5 | 3.50 | 0.5 | 3.50 | 0.5 |
| Magnesium stearate | 3.50 | 0.5 | 3.50 | 0.5 | 3.50 | 0.5 |

Hydropropyl methylcellulose sold under the trademark Methocel® is used as a hydrophilic matrix agent. The compound Methocel K15 M (trade name) is also known as hypromellose which is a hydrophilic polymer. The viscosity of an aqueous solution in water for 2% (w/w) of the compound Methocel K 15M is about 15000 mPa·s, the grade K (methoxy and hydroxypropy content) is preferred for a better hydratation rate of the polymer.

Polymers sold under the trademark Carbopol are crosslinked acrylic acid-based polymers and are used as a hydrophilic matrix agent. The compound Carbopol 71 G is polyacrylic acid polymers crosslinked, also known as Carbomer (Ph.Eur.), and Carbomer 941 (USP).

Compound sold under the trademark Kollidon is polyvinylpyrrolidone or povidone. The compound Kollidon SR comprises polyvinyl acetate (80% w/w=hydrophilic polymer), povidone (19% w/w=hydrophilic polymer) and about 0.8% sodium laurylsulfate and about 0.6% of silica.

Anhydrous colloidal silica is sold under the trademark Aerosil 200.

Tablets A, B and C release the active over a period of 24 hours and are bioequivalent (Table 2).

TABLE 2
Main pharmacokinetic parameters for tablets A, B, C and for the immediate
release

| | tmax | Cmax | AUC(0-t) | AUC | t½ | | C12 | C24 |
|---|---|---|---|---|---|---|---|---|
| Treatment | (h) | (μg/mL) | (μg * h/mL) | (μg * h/mL) | (h) | Frel | (μg/mL) | (μg/mL) |
| Immediate | 0.75 | 12.6 | 112 | 117 | 7.6 | — | 3.35 | 1.30 |
| release | | | | | | | | |
| Tablet A | 4.00 | 5.7 | 106 | 114 | 8.2 | 0.98 | 3.98 | 2.08 |
| Tablet B | 4.00 | 6.4 | 107 | 114 | 7.7 | 0.97 | 4.13 | 1.82 |
| Tablet C | 4.00 | 5.7 | 102 | 110 | 8.1 | 0.94 | 4.00 | 1.91 |

Immediate release tablet = tablet which does not content specific excipients used to obtain a sustained or controlled release of the drug
Tmax = the time necessary to obtain the plasma maximum concentration after administration of the drug
Cmax = the plasma maximum concentration observed after administration of the drug
AUC(0-t) = area under the curve (drug concentration vs time) from time 0 to time t
AUC = total area under the curve
t½ = biological half-life: the time required for half the quantity of drug deposited in a living organism to be metabolized or eliminated by normal biological process
Frel = relative bioavailability: measure of the bioavailability of the drug when compared with the immediate release formulation
C12 = plasma concentration 12 hours after the administration of the drug
C24 = plasma concentration 24 hours after the administration of the drug
* = multiplication sign

The in vitro dissolution profiles in water of tablets A, B and C were determined according to the USP 24 (apparatus n° 2, 100 rpm, aqueous medium 900 mL) over an interval of time of 12 h.

The percentages of dissolution were in the following ranges (Table 3).

TABLE 3
Percentages of dissolution of levetiracetam
obtained from tablets A, B and C.

| Time | Percentages |
|---|---|
| (h) | of dissolution |
| 0.5 | 19 ± 4 |
| 1 | 27 ± 7 |
| 2 | 42 ± 8 |
| 4 | 64 ± 10 |
| 8 | 85 ± 10 |
| 12 | 98 ± 9 |

Consequently, all the formulations owing dissolution profile similar to the results shown in Table 3 should be bioequivalent to tablets A, B and C.

### Example 2

### Formulations with a 12 h In Vitro Release of Levetiracetam in the Ranges of Tablets A, B and C as Described in Example 1

Tablets D₁ to D₁₃ are prepared by direct compression process with the following core compositions (Table 4).

TABLE 4
Core compositions of tablets D₁ to D₁₃

| Components | mg | % | mg | % | mg | % |
|---|---|---|---|---|---|---|
| | Tablet D₁ | | Tablet D₂ | | Tablet D₃ | |
| Levetiracetam | 500.00 | 62.5 | 500.00 | 50.0 | 500.00 | 71.4 |
| Methocel K 100 MCR | 292.00 | 36.5 | 492.00 | 49.2 | 193.00 | 27.6 |
| Aerosil 200 | 4.00 | 0.5 | 4.00 | 0.4 | 3.50 | 0.5 |
| Magnesium stearate | 4.00 | 0.5 | 4.00 | 0.4 | 3.50 | 0.5 |
| | Tablet D₄ | | Tablet D₅ | | Tablet D₆ | |
| Levetiracetam | 500.00 | 62.5 | 500.00 | 71.4 | 500.00 | 62.4 |
| Methocel K15 MCR | 288.00 | 36.0 | — | — | — | — |
| Methocel K 100 MCR | — | — | 96.50 | 13.8 | 146.00 | 18.3 |
| Kollidon SR | — | — | 96.50 | 13.8 | 146.00 | 18.3 |
| Aerosil 200 | 4.00 | 0.5 | 3.50 | 0.5 | 4.00 | 0.5 |
| Magnesium stearate | 8.00 | 1.0 | 3.50 | 0.5 | 4.00 | 0.5 |
| | Tablet D₇ | | Tablet D₈ | | Tablet D₉ | |
| Levetiracetam | 500.00 | 71.4 | 500.00 | 71.4 | 500.00 | 71.4 |
| Methocel K15 MCR- | — | — | 96.50 | 13.8 | 193.00 | 27.6 |
| Methocel K 100 MCR | 57.90 | 8.3 | — | — | — | — |
| Kollidon SR | 135.10 | 19.3 | 96.50 | 13.8 | — | — |
| Aerosil 200 | 3.50 | 0.5 | 3.50 | 0.5 | 3.50 | 0.5 |
| Magnesium stearate | 3.50 | 0.5 | 3.50 | 0.5 | 3.50 | 0.5 |
| | Tablet D₁₀ | | Tablet D₁₁ | | Tablet D₁₂ | |
| Levetiracetam | 500.00 | 71.4 | 500.00 | 49.0 | 500.00 | 71.4 |
| Methocel K15 MCR | 158.00 | 22.6 | 510.20 | 50.0 | 144.75 | 20.7 |
| Methocel K 100 MCR | — | — | — | — | — | — |
| Precirol ATO 5 | 35.00 | 5.0- | — | — | 48.25 | 6.9 |
| Aerosil 200 | 3.50 | 0.5 | 5.10 | 0.50 | 3.50 | 0.5 |
| Magnesium stearate | 3.50 | 0.5 | 5.10 | 0.50 | 3.50 | 0.5 |

Tablet D₁₃

| Components | mg | % |
|---|---|---|
| Levetiracetam | 500.00 | 71.4 |
| Methocel K15 MCR | 96.50 | 13.8 |
| Methocel K 100 MCR | — | — |
| Precirol ATO 5 | 96.50 | 13.8 |
| Aerosil 200 | 3.50 | 0.5 |
| Magnesium stearate | 3.50 | 0.5 |

The compound sold under the trademark Precirol® is glyceryl palmitostearate (1,2,3-propanetriol hexadecanoate octadecanoate) and is used as lipohilic and hydrophobic matrices.

The in vitro dissolution profiles in water of tablets D₁ to D₁₃ were determined according to the USP 24 (apparatus n° 2, 100 rpm, aqueous medium 900 mL) over an interval of time of 12 h.

All the percentages of dissolution were in the ranges of the Table 3 (example 1).

### Example 3

### Coated Tablet with a 24 h In Vivo Release of Levetiracetam

Table 6 shows a pharmaceutical composition F which was manufactured by dry granulation process.

TABLE 6
Composition of the coated tablet F

| Components | quantities in mg |
|---|---|
| Levetiracetam | 500.00 |
| Hydroxypropylmethylcellulose | 187.75 |
| Macrogol 6000 | 7.00 |
| Anhydrous colloidal silica | 3.50 |
| Magnesium stearate | 1.75 |
| Opadry ® 85F18422 white | 21.00 |

The in vitro dissolution profiles in water of tablet F was determined according to the USP 24 (apparatus n° 2, 100 rpm, aqueous medium 900 mL) over an interval of time of 12 h (Table 6).

TABLE 6
Percentages of dissolution of levetiracetam from tablet F.

| Time | Percentages |
|---|---|
| (h) | of dissolution |
| 0.5 | 21 |
| 1 | 33 |
| 2 | 50 |
| 4 | 72 |
| 8 | 94 |
| 12 | 100 |

All the percentages of dissolution were in the ranges of the Table 3 (example 1).

### Example 4

Table 7 shows two pharmaceutical compositions G and H which were manufactured by dry granulation process.

TABLE 7
Composition of the coated tablets G and H

| | |
|---|---|
| Tablet G | Tablet H |

| | |
|---|---|
| Components | quantities in mg |

| | | |
|---|---|---|
| Levetiracetam | 1000.00 | 750.00 |
| Hydroxypropylmethhylcellulose | 375.50 | 281.60 |
| Macrogol 6000 | 14.00 | 10.50 |
| Anhydrous colloidal silica | 7.00 | 5.25 |
| Magnesium stearate | 3.50 | 2.65 |
| Opadry ® 85F18422 white | 42.00 | 31.50 |

The in vitro dissolution profiles in water of tablets G and H was determined according to the USP 24 (apparatus n° 2, 100 rpm, aqueous medium 900 mL) over an interval of time of 12 h. All the percentages of dissolution were in the ranges of the Table 3.

### Example 5

Tablet I was prepared by direct compression process according to the invention with the following core compositions (Table 7).

TABLE 7
Composition of the tablet I

| Components | quantities in mg |
|---|---|
| Levetiracetam | 500.00 |
| Methocel K15M CR | 369.38 |
| Kollidon SR | 123.13 |
| Anhydrous colloidal silica | 5.00 |
| Magnesium stearate | 2.50 |

All the percentages of dissolution were in the ranges of the Table 3.

### Example 6

Tablets J and K were prepared by direct compression process according to the invention with the following core compositions (Table 8).

TABLE 8
Composition of the tablets J and K

| | |
|---|---|
| Tablet J | Tablet K |

| | |
|---|---|
| Components | quantities in mg |

| | | |
|---|---|---|
| Levetiracetam | 500.00 | 500.00 |
| Methocel K15M CR | 186.00 | 158.00 |
| Anhydrous colloidal silica | 3.50 | 3.50 |
| Magnesium stearate | 3.50 | 3.50 |
| PEG 6000 | 7.00 | 35.00 |

All the percentages of dissolution were in the ranges of the Table 3.

### Example 7

Tablet L was prepared by direct compression process with the following core composition (Table 9).

| | Tablet L | |
|---|---|---|
| Components | Quantities in mg | % |
| Levetiracetam | 500.00 | 50.0 |
| Methocel K 15 MCR | 369.40 | 36.9 |
| Pevikon P737P | 123.10 | 12.3 |
| Aerosil 200 | 5.00 | 0.5 |
| Magnesium stearate | 2.50 | 0.3 |

Compound sold under the trademark Pevikon is a PVC resin.

All the percentages of dissolution were in the ranges of the Table 3. So the formulation owing dissolution profile similar to the results shown in Table 3 is bioequivalent to tablets A, B and C.

1. A pharmaceutical composition in the form of a tablet comprising 500 mg of Levetiracetam, 193.00 mg of a hydroxypropyl methylcellulose, 3.5 mg of anhydrous colloidal silica and 3.50 mg of magnesium stearate.

2. A pharmaceutical composition in the form of a tablet comprising 500 mg of Levetiracetam, 96.50 mg of a hydroxypropyl methylcellulose, 96.50 mg of a mixture comprising polyvinylacetate and polyvinylpyrrolidinone, 3.5 mg of anhydrous colloidal silica and 3.50 mg of magnesium stearate.

3. A pharmaceutical composition in the form of a tablet comprising 500 mg of Levetiracetam, 158.00 mg of a hydroxypropyl methylcellulose, 35 mg of a cross-linked acrylic acid based polymer, 3.5 mg of anhydrous colloidal silica and 3.50 mg of magnesium stearate.

4. A pharmaceutical composition in the form of a tablet comprising 500 mg of Levetiracetam, 187.75 mg of a hydroxypropyl methylcellulose, 7 mg of a polyethylene glycol (PEG), 3.5 mg of an anhydrous colloidal silica and 1.75 mg of magnesium stearate.

5. A pharmaceutical composition according to any of claims 1 to 4 which is coated with a polyvinyl alcohol.

6. Use of a pharmaceutical composition according to any of claims 1 to 5 for the treatment of epilepsy.
